(19) United States
(12) Patent Application Publication
(10) Pub. No.: US 2005/0106194 A1
(43) Pub. Date: May 19, 2005

(54) Compositions for achieving benefits in skin using key cellular metabolic intermediates

(75) Inventor: John R. Schiltz, Coppell, TX (US)

Correspondence Address: FULBRIGHT & JAWORSKI L.L.P., 600 CONGRESS AVE., SUITE 2400, AUSTIN, TX, 78701, US

(73) Assignee: Mary Kay Inc.

(21) Appl. No.: 10/716,378

(22) Filed: Nov. 18, 2003

(51) Int. Cl. A61K 7/025; A61K 7/00

(52) U.S. Cl. 424/401; 424/64

(57) ABSTRACT

The present invention relates to novel methods and compositions comprising key metabolic ingredients for treating aged, mature, nutritionally-compromised, or environmentally-damaged skin. These methods and compositions provide improvements in the skin's visual appearance, physiological functions, clinical properties, and biophysical properties.

### BACKGROUND OF THE INVENTION

A. Field of the Invention

The present invention relates generally to a treatment method and composition for improving the skin's visual appearance, function, and/or clinical/biophysical properties which have been changed by factors such as chronological age, chronic sun exposure, adverse environmental conditions and factors such as pollutants, household chemicals, disease pathologies, smoking, and/or malnutrition. In particular, the present invention is directed towards compositions and methods for their use comprising a combination of naturally-occurring key metabolic ingredients that can normalize abnormal metabolism in skin cells.

B. Background of the Invention

With chronological age, chronic exposure to adverse environmental factors, or malnutrition, the visual appearance, physical properties, and physiological functions of skin change in ways that are considered cosmetically undesirable. The most notable and obvious changes include the development of fine lines and wrinkles, loss of elasticity, increased sagging, loss of firmness, loss of skin clarity or color evenness, coarse surface texture, and mottled pigmentation. Less obvious, but measurable changes which occur as skin ages or endures chronic environmental insult include a general reduction in cellular and tissue vitality, reduction in cell replication rates, reduced cutaneous blood flow, reduced moisture content, accumulated errors in structure and function, alterations in the normal regulation of common biochemical pathways, and a reduction in the skin's ability to remodel and repair itself. Many of the alterations in appearance and function are caused by changes in the outer epidermal layer of the skin, while others are caused by changes in the lower dermis.

Several different approaches can be used to treat aged or environmentally-damaged skin, or skin that is unhealthy due to malnutrition. One approach involves the use of specific agents to directly stimulate or inhibit selected biochemical targets. Examples include the use of retinoids to stimulate collagen and glycosaminoglycan synthesis by fibroblasts (Schiltz et al., 1986). Another approach is to use agents or processes that stimulate the rate at which the epidermis replaces itself, a process known as epidermal cell renewal. Increases in epidermal cell renewal rates usually result from a more rapid rate of replication of epidermal basal cells, and can be caused by diverse stimuli such as chemical or physical injury, adverse environmental conditions, or direct stimulators of basal cell division.

Some examples of chemical injury include allergic or non-allergic contact irritation, pH extremes, or interaction of the stratum corneum with household or industrial chemicals or pollutants. Physical injury can include skin abrasion, friction (i.e. on the soles and heels of the feet), or removal of the stratum corneum by physical exfoliation (i.e. cosmetic masks) or by tape stripping. Agents that directly or indirectly stimulate basal cell division include hydroxy acids, retinoids, or barrier disrupters. For example, U.S. Pat. No. 5,720,963 discloses that a combination of hydroxy acids, retinoids, and cerebrosides causes chronic injury to the stratum corneum and results in epidermal and dermal repair of the structurally-deteriorated skin. U.S. Pat. No. 6,495,126, for example, uses a combination of surfactants and chelating agents to stimulate an endogenous stratum corneum chymotryptic proteinase that causes a loosening of corneocytes, resulting in an increased rate of epidermal replacement and chronic anti-aging benefits. Adverse environmental exposures that can result in more rapid epidermal turnover rates include UVA, UVB, and IR radiation from the sun and cold coupled with low relative humidity (i.e. low dew point).

Many of the above methods of increasing stratum corneum renewal rates have various drawbacks, such as significant irritation to the skin, skin toxicity, or low pH. In addition, most of these methods involve the invocation of chronic damage to the skin, which sets up repair mechanisms. For most of the existing treatments, there will be a period of time, up to several weeks or months, during which the skin becomes irritated and after which tolerance sets in and the symptoms of irritation may decrease and/or cease.

### SUMMARY OF THE INVENTION

The present invention overcomes the deficiencies in the art by providing compositions and methods for their use that can be used to treat aged, mature, nutritionally-compromised, or environmentally-damaged skin.

The present invention includes methods and compositions for treating or preventing aged or damaged skin. Damaged skin can include nutritionally compromised skin or environmentally damaged skin. Environmentally damaged skin can include skin damaged by u.v. light, chronic sun exposure, environmental pollutants, chemicals, disease pathologies, and/or smoking.

In particular embodiments, the compositions of the invention include at least one regulator of lipid metabolism; at least one regulator of polysaccharide metabolism; at least one regulator of cellular protein metabolism; and at least one regulator of nucleic acid metabolism. The composition can be formulated to be chemically compatible. The composition can also be formulated as a cosmetic mixture or compound or comprised in a cosmetic vehicle. In particular embodiments, the cosmetic vehicle includes an emulsion, a cream, a lotion, a solution, an anhydrous base, a gel, or an ointment. The emulsion can be an oil in water emulsion or a water in oil emulsion. The solution can be an aqueous solution or hydro-alcoholic solution. The anhydrous base can be a lipstick or a powder. The composition can also be included in an anti-aging product or a moisturizing product, or in any product designed to provide benefit to the skin.

In other embodiments, the composition is adapted for application at least once a day during use. The composition can also be adapted for application at least twice a day, three times a day, four times a day, five times a day or more during use.

In certain aspects of the present invention, at least one regulator of lipid metabolism can be selected from the group consisting of sodium citrate, linoleic acid, linolenic acid, biotin, glucose, sodium acetate, mevalonic acid, and serine, or a derivative thereof. At least one regulator of polysaccharide metabolism can be selected from the group consisting of galactosamine, glucosamine, xylose, and magnesium chloride, or a derivative thereof. At least one regulator of cellular protein metabolism can be an amino acid, or a derivative thereof. The amino acid can be an essential or non-essential amino acid, or derivatives thereof. The non-essential amino acid can be selected from the group consisting of arginine, histidine, isoleucine, leucine, lysine, methionine, phenylalanine, threonine, tryptophan, and valine, or a derivative thereof. Other amino acids that can be used with the present invention include, for example, serine, aspartic acid, glutamic acid, asparagine, glutamine, alanine, tyrosine, cysteine, glycine, and proline, or derivatives thereof. At least one regulator of nucleic acid metabolism can be selected from the group consisting of sodium bicarbonate, aspartic acid, sodium phosphate, niacin, glutamine, and glucose, or a derivative thereof.

In particular embodiments of the present invention, the composition can comprise from about 0.001% to about 5.0% of at least one regulator of lipid metabolism, polysaccharide metabolism, cellular protein metabolism, and/or nucleic acid metabolism.

The terms “mixture,” “mix,” and “mixing” or any variants of these terms, when used in the claims and/or specification includes, stirring, blending, dispersing, milling, homogenizing, and other similar methods. The mixing of the components or ingredients of the disclosed compositions can form into a solution. In other embodiments, the mixtures may not form a solution. The ingredients/components can also exist as undissolved colloidal suspensions.

The terms “inhibiting,” “reducing” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve the methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

Aged, nutritionally-compromised, and environmentally-damaged skin affects many people in today society. Fine lines, wrinkles, loss of elasticity, increased sagging, loss of firmness, loss of color eveness, course surface texture, and mottled pigmentation are just some examples of some of the attributes of damaged skin. Previous attempts to treat damaged skin have various drawbacks ranging from skin irritation to skin toxicity. The present invention is an effective alternative to the use of hydroxy acids, retinoid compounds, botanical extracts, or other materials currently used to treat aged or environmentally-damaged skin.

The present invention discloses novel methods and compositions for treating damaged skin. The methods and compositions disclosed in this specification provide treatments that can improve the skin's visual appearance, physiological functions, clinical properties, and biophysical properties by stimulating and/or activating skin cells to normalize and improve their metabolism. Chronic stimulation is effective to cause improvements in the repair and replacement of the stratum corneum, epidermis, and dermis of the skin, which results in improvements in the appearance, function and physical properties of the aged, nutritionally-compromised, or environmentally-damaged skin. These and other aspect of the present invention are described in further detail below.

A. Key Metabolic Ingredients

Eukaryotic cells, including skin cells, contain thousands of distinct molecules. These molecules can be categorized into four major classes: proteins, lipids, carbohydrates, and nucleic acids. In addition, chemical or physical combinations of all these molecules exist in cells. For example, lipoproteins, glycoproteins, glycolipids, and nucleoproteins, all of which are constructed from combinations of the four major classes. Skin cells function optimally when the correct balance between the amounts and types of molecules is reached.

The primary source for the key small molecules (or key metabolic ingredients) that serve as precursors to proteins, lipids, carbohydrates, and nucleic acids is from the diet or from the catabolism and mobilization of storage reserves from fat or complex carbohydrates. If the supply of the key metabolic ingredients is reduced or if there is an imbalance in the amounts and types of such ingredients, skin cells will not perform optimally. The reduction or imbalance of key metabolic ingredients in skin cells can be caused by, for example, poor circulation, malnutrition, aging, environmental changes, chemical injury, or physical injury. This, in turn, can result in impaired physiological functions and subsequent changes in the skin's physical properties and clinical appearance.

Key metabolic ingredients can normalize the abnormal metabolism in skin cells that result in abnormal accumulation in the amounts and types of skin lipids, polysaccharides, proteins, and nucleic acids. The can also serve as metabolic precursors to increase the cell's energy supply, which is needed to repair and remodel aged or environmentally-damaged skin cells and tissues. Key metabolic ingredients that can be used with this invention, therefore, can be classified into four groups: (1) regulators of lipid metabolism; (2) regulator of polysaccharides; (3) regulators of cellular proteins; and (4) regulators of nucleic acids. Table 1 includes non-limiting examples of regulators of lipid metabolism that can be used with the present invention. Table 2 includes non-limiting examples of regulators of polysaccharides that can be used with the present invention. Table 3 includes non-limiting examples of regulators of cellular proteins that can be used with the present invention. Table 4 includes non-limiting examples of regulators of nucleic acids that can be used with the present invention.

TABLE 1
Regulators of Lipid Metabolism

| Regulators of | |
|---|---|
| Lipid Metabolism | Beneficial Function In Skin |
| Sodium Citrate | Stimulates fatty acid synthesis to plump and |
| | soften aged skin |
| Linoleic and Linolenic | These are essential fatty acids required for |
| Acids | barrier ceramide synthesis and for the formation |
| | of unsaturated fatty acids such as arachidonic |
| | acid, which is involved in cellular |
| | communication |
| Biotin | A B-complex vitamin required to activate acetyl |
| | CoA carboxylase, a key enzyme for fatty acid |
| | synthesis |
| Glucose | The simple blood sugar which is metabolized to |
| | form cellular energy and acetyl CoA, which is a |
| | precursor to all the lipids (fatty acids, steroids, |
| | ceramides, phospholipids), proteins (via |
| | formation of amino acids), and polysaccharides |
| | (glycogen, glycosaminoglycans) |
| Sodium Acetate | A direct precursor to Acetyl CoA, a precursor to |
| | all cellular lipids |
| Mevalonic Acid | This ingredient is rate limiting in the formation |
| | of steroids such as cholesterol, a critical barrier |
| | lipid and an important component of all living |
| | cell membranes |
| Serine | An amino acid that reacts with palmitoyl CoA to |
| | form sphingosine, a direct precursor to the |
| | barrier sphingolipids. It is also a precursor to |
| | phospholipids, which are an important part of all |
| | living cell membranes |

TABLE 2
Regulators of Polysaccharide Metabolism

| Regulators of | |
|---|---|
| Polysaccharides | Beneficial Function In Skin |
| Galactosamine and | These ingredients are metabolic precursors to all |
| Glucosamine | glycosaminoglycans such a hyaluronic acid, |
| | chondroitin sulfate, keratin sulfate, and dermatan |
| | sulfate, all of which are important for skin |
| | firming, wrinkle reduction, and deep hydration |
| | of the skin |
| Xylose | Important for the formation of proteoglycans, |
| | which are combinations of proteins and |
| | polysaccharides that are important for skin |
| | elasticity and firmness, wrinkle reduction, and |
| | deep hydration |
| Magnesium Chloride | Required for all kinase reactions involved in the |
| or Mg⁺⁺ | formation and utilization of adenosine |
| | triphosphate (ATP), which is needed to produce |
| | UDP-sugars, which are the direct precursors to |
| | polysaccharides, including glycosaminoglycans. |
| | Mg⁺⁺ is also important for the synthesis of |
| | nucleic acids, and for general energy formation |
| | and utilization within cells |

TABLE 3
Regulators of Cellular Protein Metabolism

| Regulators of | |
|---|---|
| Cellular Proteins | Beneficial Function In Skin |
| Essential Amino Acids | Of the 21 amino acids that comprise proteins, 10 |
| | are essential because they cannot be synthesized |
| | by humans. These must be included in the diet. |
| | In nutritionally-compromised or aged skin, these |
| | can be rate limiting for the formation of proteins, |
| | which include all the enzymes and most of the |
| | complex structures of cells and tissues. The |
| | essential amino acids include Arginine, |
| | Histidine, Isoleucine, Leucine, Lysine, |
| | Methionine, Phenylalanine, Threonine, |
| | Tryptophan, and Valine |
| Non-Essential Amino | These include Glutamine (which is needed to |
| Acids | synthesize all the non-essential amino acids); |
| | Glycine (which comprises about 25% of the |
| | amino acid residues of collagen, and a main |
| | amino acid of filaggrin, which is a precursor to |
| | natural moisturization factor); Proline (which |
| | comprises about 25% of the amino acid residues |
| | of collagen, and a main amino acid component of |
| | filaggrin); Glutamic acid (a main amino acid |
| | residue in filaggrin); and Cysteine (which is |
| | important for the formation of disulfide bonds of |
| | keratin, the protein that forms the major |
| | structural component of the epidermis and |
| | stratum corneum) |

TABLE 4
Regulators of Nucleic Acids (DNA and RNA)

| Regulators of | |
|---|---|
| Nucleic Acids | Beneficial Function In Skin |
| Sodium Bicarbonate | Forms carbon dioxide which reacts with |
| | glutamine and ATP to form carbamoyl |
| | phosphate. This represents a major regulatory |
| | step in the synthesis of the pyrimidine bases that |
| | are essential building blocks for both DNA and |
| | RNA |
| Aspartic Acid | A non-essential amino acid that reacts with |
| | carbamoyl phosphate to commit the cell to the |
| | synthesis of the pyrimidine bases |
| Sodium Phosphate | A source of phosphate, which is a major |
| | component of both DNA and RNA |
| Niacin | A vitamin that forms an important redox |
| | ingredient called nicotinamide adenine |
| | dinucleotide phosphate (NADP). NADP is rate |
| | limiting in the formation of ribose phosphate, a |
| | major component of nucleic acids. Nicotinamide |
| | or its salts or esters can also be used, which have |
| | the added benefit of skin vasodilatation |
| Glutamine | A non-essential amino acid that combines with a |
| | ribose sugar phosphate derivative. Glutamine is |
| | the commitment step to the synthesis of the |
| | purine bases that are essential building blocks of |
| | both DNA and RNA |
| Glucose | A key precursor for the formation of the pentose |
| | phosphate sugars that are important for the |
| | synthesis of DNA and RNA. Glucose is also |
| | important for the generation of cellular energy, |
| | which is needed for building proteins, lipids, and |
| | polysaccharides |

B. Compositions of the Present Invention

A person of ordinary skill would recognize that the present compositions must include at least one key metabolic ingredient. In other non-limiting embodiments, for example, the compositions can include at least two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty or more key metabolic ingredients.

In certain non-limiting embodiments, the present compositions may comprise in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% of at least one key metabolic ingredient, and any range derivable therein. A person of ordinary skill in the art would understand that the concentrations for key metabolic ingredients in a composition can vary depending on the addition, substitution, and/or subtraction of additional key metabolic ingredients. Moreover, a person of ordinary skill in the art would recognize that the key metabolic ingredients in a composition can vary by the addition, subtraction and/or substitution with other compounds, including other similar key metabolic ingredients.

The disclosed compositions of the present invention may also include various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

C. Cosmetic Vehicles

The present compositions are effective in all types of cosmetic vehicles. Non-limiting examples of suitable cosmetic vehicles include emulsions, creams, lotions, solutions (both aqueous and hydro-alcoholic), anhydrous bases (such as lipsticks and powders), gels, and ointments or by other method or any combination of the forgoing as would be known to one of ordinary skill in the art (Remington's, 1990). Variations and other appropriate vehicles will be apparent to the skilled artisan and are appropriate for use in the present invention.

In preferred embodiments, the cosmetic vehicle is selected from oil-in-water emulsions, hydro-alcoholic solutions, and encapsulated beads in anhydrous systems. With respect to oil-in-water emulsions, such emulsions and their compositions and methods of making are well known in the art. It is important, however, that the concentrations and combinations of the key metabolic ingredients be selected in such a way that the combinations are chemically compatible and do not form complexes which precipitate from the finished product.

D. Cosmetic Products

The composition of the present invention can be used in many cosmetic products including, but not limited to, moisturizing cream, skin benefit creams and lotions, gels, ointments, foundation, night cream, lipstick, cleansers, toners, masks, and color cosmetic products. The composition is most preferably used in anti-aging products for the face and other body parts, most especially leave-on products.

E. Additional Compounds and Agents that Can be Used in Combination with the Present Compositions

Compositions of the present invention can include other beneficial agents and compounds such as, for example, acute or chronic moisturizing agents (including, e.g., humectants, occlusive agents, and agents that affect the natural moisturization mechanisms of the skin), anti-oxidants, sunscreens having UVA and/or UVB protection, skin lightening agents (e.g. hydroquinone), hydroxy acids, emollients, anti-irritants, vitamins, trace metals, anti-microbial agents, botanical extracts, fragrances, and/or dyes and color ingredients.

1. Moisturizing Agents

Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturization factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, acrylates/C10-30 alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus armeniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, beta-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays)oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, tructose, gelatin, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesquistearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quaternium-18 hectorite, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, salicylic acid, sandalwood (santalum album) oil, serine, serum protein, sesame (sesamum indicum) oil, shea butter (butyrospermum parkii), silk powder, sodium chondroitin sulfate, sodium DNA, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

2. Antioxidants

Non-limiting examples of antioxidants that can be used with the compositions of the present invention include acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

3. Compounds Having Ultraviolet Light Absorbing Properties

Non-limiting examples of compounds that have ultraviolet light absorbing properties that can be used with the compounds of the present invention include benzophenone, benzophenone-1, benzophenone-2, benzophenone-3, benzophenone-4 benzophenone-5, benzophenone-6, benzophenone-7, benzophenone-8, benzophenone-9, benzophenone-10, benzophenone-11, benzophenone-12, benzyl salicylate, butyl PABA, cinnamate esters, cinoxate, DEA-methoxycinnamate, diisopropyl methyl cinnamate, ethyl dihydroxypropyl PABA, ethyl diisopropylcinnamate, ethyl methoxycinnamate, ethyl PABA, ethyl urocanate, glyceryl octanoate dimethoxycinnamate, glyceryl PABA, glycol salicylate, homosalate, isoamyl p-methoxycinnamate, PABA, PABA esters, Parsol 1789, and isopropylbenzyl salicylate.

4. Additional Compounds and Agents

Non-limiting examples of additional compounds and agents that can be used with the compositions of the present invention include skin lightening agents (e.g. kojic acid, hydroquinone, ascorbic acid and derivatives, retinoids and their derivatives, and niacinamide), hydroxy acids (e.g. alpha and beta hydroxy acids and polymeric hydrox acids), emollients (e.g. esters and fatty acids), vitamins (e.g. D, E, A, K, and C), trace metals (e.g. zinc, calcium and selenium), anti-irritants (e.g. steroids and non-steroidal anti-inflammatories), antimicrobial agents (e.g. triclosan), botanical extracts (e.g. aloe vera, chamomile, cucumber extract, ginkgo bibloba, ginseng, and rosemary), dyes and color ingredients (e.g. D&C blue no. 4, D&C green no. 5, D&C orange no. 4, D&C red no. 17, D&C red no. 33, D&C violet no. 2, D&C yellow no. 10, D&C yellow no. 11 and DEA-cetyl phosphate), preservatives (e.g. BHA), emollients (i.e. organic esters, fatty acids, lanolin and its derivatives, plant and animal oils and fats, and di- and triglycerides), antimicrobial agents (e.g., triclosan and ethanol), and fragrances (natural and artificial).

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### A Non-Limiting Example of a Specific KMI Composition

A non-limiting example of one embodiment of the present invention is exhibited in Table 5. The ingredients in Table 5 were selected based on their abilities to affect the content and balance of the four groups of important macromolecules or hybrid molecules known to be important for normal health and optimal functioning of skin. The specific mixture of the ingredients in Table 5 are referred to as the “key metabolic intermediates” blend, or KMI blend. The percentages that are referred to in Table 5 constitute a “1×” concentration of the KMI blend. If the concentration of the blend is used in half the amount, the concentration of the blend is referred to as “0.5×”, and if used at twice the concentration, the concentration is referred to as “2×”, etc. As a person of ordinary skill in the art would recognize, the key metabolic ingredients in Table 5 can be eliminated, and/or substituted with other ingredients. By way of example only, sodium citrate can be replaced with potassium citrate, free citric acid, or esters of citric acid. Linoleic and linolenic acids can be replaced with, for example, salts or esters of these acids. Galactosamine, for example, can be substituted with glucosamine because galactosamine can be generated from the action of cellular epimerases on glucosamine. Mevalonic acid is optional, although it is expected that leaving the material out will result in slightly reduced chronic anti-aging activity. Addition of vasodilating materials such as nicotinamide or niacin will stimulate blood flow, which will improve the activity of the mixture. Anyone skilled in the knowledge of cellular intermediary metabolism will be aware of appropriate substitutions in chemical forms. These substitutions, for example, can be based on the inter-conversions of one biochemical substance to another through established biochemical pathways.

TABLE 5

| Ingredient | % In Final Formula |
|---|---|
| Arginine | 0.1875 |
| Aspartic Acid | 0.1250 |
| Biotin | 0.0025 |
| Cysteine | 0.1250 |
| Galactosamine | 0.0250 |
| Glucosamine | 0.0625 |
| Glucose | 0.2500 |
| Glutamic Acid | 0.1250 |
| Glutamine | 0.2500 |
| Glycine | 0.1250 |
| Histidine | 0.1250 |
| Isoleucine | 0.1250 |
| Leucine | 0.1250 |
| Linoleic Acid, Na⁺ Salt | 0.0250 |
| Linolenic Acid, Na⁺ Salt | 0.0250 |
| Lysine | 0.1250 |
| Methionine | 0.1250 |
| Mevalonic Acid | 0.0250 |
| MgCl₂.6H₂O | 0.0250 |
| NaH₂PO₄.2H₂O | 0.1250 |
| NaHCO₃ | 0.0625 |
| Phenylalanine | 0.1250 |
| Proline | 0.1250 |
| Serine | 0.1250 |
| Sodium Acetate.3H2O | 0.2000 |
| Sodium Citrate.2H2O | 0.1000 |
| Threonine | 0.1250 |
| Tryptophan | 0.1250 |
| Valine | 0.1250 |
| Xylose | 0.1250 |

### Example 2

### Materials and Methods

The following procedure was used to determine stratum corneum transit time in human subjects. Determination of stratum corneum transit time is an indirect measurement of epidermal cell activation, and a good predictor of chronic anti-aging benefits on the skin.

As many as 6 different sites per forearm were marked using a plastic template, and baseline readings of color intensity were determined using a Minolta Chromameter (b* value). Occlusive Hilltop chambers (2 cm diameter) containing 0.05 ml Mary Kay Sun Essentials® Sunless Tanning Lotion that contains dihydroxy acetone (DHA) were placed on the sites. After 6 hours, these patches were removed, and 18 hours later, the color intensity was again determined using the Chromameter. The delta b* (Δb*) values were calculated as the difference between the reading and the baseline. Panelists themselves applied the products to the brown spots in the morning and evening during the ensuing 10 days, and the Chromameter readings were repeated after 3, 5, 7, and 10 days. The color decay slope was calculated as the percent color loss per day, and the stratum corneum transit time determined by extrapolating to 100% loss of color.

The activity of the KMI blend from Table 5 is not dependent on the vehicle, as long as the vehicle is a suitable carrier of the KMI components to the surface of the skin. For the experiments to be described, three different vehicles were used, which are referred to as vehicles A, B, and C. Vehicle A (Table 6) is a simple non-moisturizing, non-drying oil-in-water emulsion (75% water) which is used to dissolve hydrophobic and/or hydrophilic ingredients for testing on the skin. Vehicle B (Table 7) is a hydroalcohol Gel containing 49.5% water+49.5% ethanol+1% Keltrol CR. Vehicle C (Table 8) is a proprietary, highly-moisturizing oil-in-water emulsion that is modified from a marketed Mary Kay moisturizer.

TABLE 6
Composition of Vehicle A

| Phase | Ingredient | % In Formula |
|---|---|---|
| A | Water | 86.44 |
| A | Xanthan gum | 0.1 |
| B | Methyl Paraben | 0.15 |
| B | Propyl Paraben | 0.1 |
| B | Citric Acid | 0.01 |
| C | Cetyl Alcohol | 4.0 |
| C | Glycerol Stearate | 4.0 |
| C | Octyl Palmitate | 1.0 |
| C | Tocopheryl Acetate | 0.2 |

Procedure to make Vehicle A: Disperse Phase A at room temperature with stirring, and heat to 75° C. Add Phase B and disperse completely. Heat to 75° C., and combine with Phase C. Allow to cool, with mixing to 30° C.

TABLE 7
Composition of Vehicle B

| Phase | Ingredient | % In Formula |
|---|---|---|
| A | Water | 49.5 |
| A | SDA 40B Ethanol | 49.5 |
| — | Keltrol CR | 1.0 |

Procedure to make Vehicle B: Mix the water and SDA 40B at room temperature and disperse the Keltrol CR with stirring until solubilized.

TABLE 8
Composition of Vehicle C

| Phase | Ingredient | % In Formula |
|---|---|---|
| A | Water | 58.4 |
| A | Glycereth-26 | 5.0 |
| A | Hispagel | 5.0 |
| A | Disodium EDTA | 0.05 |
| A | Carbopol 940, 2% | 15.0 |
| B | Lecinol S-10 | 1.0 |
| C | Cosmowax J | 1.25 |
| C | Finsolve TN | 6.0 |
| C | Dimethicone | 0.5 |
| C | Isostearyl Alcohol | 1.25 |
| C | Cetyl Alcohol | 0.7 |
| C | Silica | 0.35 |
| D | Triethanolamine, 99% | 1.16 |
| D | Water | 1.60 |
| E | Germaben II | 1.0 |
| F | Sodium PCA | 0.11 |
| F | Prodew 400 | 0.7 |
| F | Tocopheryl Acetate | 0.1 |
| F | Phospholipid EFA | 0.82 |

Procedure to make Vehicle C: Add the ingredients in phase A to vessel, in order, at room temperature, mixing between additions. Begin heating to 75° C. At 50° C., add phase B. At 75° C. add phase C, in order, mixing between additions. As mixture cools, add phase D at 65° C. At 45° C., add phase E and phase F.

### Example 3

### Effect of KMI in Vehicle A on Stratum Corneum Turnover Time

The effects of KMI on stratum corneum turnover time, as tested in Vehicle A, is shown in Table 9. For untreated skin, corneum turnover time was estimated to be about 12.7 days, and Vehicle A reduced this time to 11.2 days, for a reduction of 11.7%. Most, if not all, vehicles will reduce transit time 10 to 12%, and this is attributed in some part to the physical action of rubbing the stratum corneum. Addition of 1× KMI reduced the transit time 12.8%, 2× KMI reduced the transit time 16.2%, and 4× KMI reduced the transit time 30.6%. Thus, the effect of KMI on transit time is concentration-dependent.

TABLE 9
Concentration Effects of KMI in Vehicle A on
Stratum Corneum Turnover Time

| | Stratum Corneum | |
|---|---|---|
| | Turnover | % Reduction in Turnover |
| Treatment | Time* (Days) | Time vs. Untreated |
| Untreated | 12.7 | — |
| Vehicle A | 11.2 | 11.7. |
| 1 × KMI in Vehicle A | 11.1 | 12.8. |
| 2 × KMI in Vehicle A | 10.6 | 16.2. |
| 4 × KMI in Vehicle A | 8.8 | 30.6 |

*The stratum corneum turnover time was measured using the DHA disappearance method during a 10-day period. Twelve panelists participated in the study, and they all were treated with all the test formulas. Vehicle A (Table 6) is a simple non-moisturizing, non-drying oil-in-water emulsion.

### Example 4

### Effect of KMI in Vehicle B on Stratum Corneum Turnover Time

The effects of KMI on stratum corneum turnover time, as tested in Vehicle B, is shown in Table 10. For untreated skin, corneum turnover time was estimated to be about 10.4 days. Vehicle B reduced this time to 9.0 days, for a reduction of 12.7%. Addition of 4× KMI reduced the transit time to 32.3%. These data show that KMI activates the epidermis when tested in Vehicle B (Table 7), which results in a decrease in the time required for stratum corneum replacement.

TABLE 10
Effects of KMI in Vehicle B on Stratum Corneum Turnover Time

| | Stratum Corneum | |
|---|---|---|
| | Turnover | % Reduction in Turnover |
| Treatment | Time* (Days) | Time vs. Untreated |
| Untreated | 10.4 | — |
| Vehicle B | 9.0 | 12.7 |
| 4 × KMI in Vehicle B | 7.0 | 32.3 |

*The stratum corneum turnover time was measured using the DHA disappearance method during a 10-day period. Twelve panelists participated in the study, and they all were treated with all the test formulas. Vehicle B (Table 7) is a hydroalcoholic gel containing 49.5% water + 49.5% SDA 40B + 1% Keltrol CR.

### Example 5

### KMI is Not an Effective Acute Moisturizer

Daily treatment of skin with an effective acute moisturizing formula can result in chronic anti-aging benefits. In order to rule out the possibility that the chronic anti-aging benefits of KMI are due to its ability to acutely moisturize the skin, 4× KMI was formulated into vehicle A and tested during a 6 hour period for its effects on skin moisture. Skin moisture was measured using the Nova Dermal Phase meter instrument. The results presented in Table 11 indicate that the untreated skin in this study became somewhat dry during the 6 hours of the experiment. Vehicle A reduced the rate at which the skin became dry, and the addition of 4× KMI in Vehicle A had no additional statistically-significant moisturization effect. In contrast, the effects of a commercial Mary Kay moisturizer increased skin moisture considerably during the 6 hours of the experiment. These data indicate that KMI is not an effective acute moisturizer.

TABLE 11
Effects of KMI in Vehicle A on Acute Moisturization of Human Skin

| | % Increase in Moisture vs. Baseline* | | |
|---|---|---|---|
| Treatment | 2 Hours | 4 Hours | 6 Hours |
| Untreated | 8.1 | −5.6 | −9.9 |
| Vehicle A | 11.6 | 3.9 | 1.6 |
| 4X KMI in Vehicle A | 13.4 | 9.3 | 1.01 |
| Mary Kay Commercial | 59.1 | 30.6 | 18.8 |
| Facial Moisturizer** | | | |

*Acute moisturization of the inner aspects of the forarms of 10 human panelists was tested during a 6-hour period using the Nova Dermal Phase Meter. After baseline readings were taken, the products were applied and measurements taken again after 2, 4, and 6 hours. Vehicle A is a simple non-moisturizing, non-drying oil-in-water emulsion.
**The Mary Kay Commercial Facial Moisturizer used is TIMEWISE ® Age-Fighting Moisturizer which can be obtained, for example, from Mary Kay, Inc.

### Example 6

### Effect of KMI in Vehicle C on Stratum Corneum Turnover Time

The effect of KMI on skin moisture during a 6 hour period, as tested in Vehicle C (Table 8), is included in Table 12. The untreated skin sites were not moisturized or dried in this study. Vehicle C demonstrated statistically-significant moisturization 2, 4, and 6 hours after product application. A 1× concentration of KMI, formulated in Vehicle C, did not increase the acute moisturization by the Vehicle. These data indicates that KMI does not possess acute moisturization properties when tested in Vehicle C.

TABLE 12
Effects of KMI in Vehicle C on Acute Moisturization of Human Skin

| | % Increase in Moisture vs. Baseline* | | |
|---|---|---|---|
| Treatment | 2 Hours | 4 Hours | 6 Hours |
| Untreated | −2.0 | 2.4 | 0.4 |
| Vehicle C | 40.5. | 41.9 | 30.8 |
| 1X KMI inVehicle C | 34.1 | 43.6 | 23.4 |

*Acute moisturization of the inner aspects of the forarms of 10 human panelists was tested during a 6-hour period using the Nova Dermal Phase Meter. After baseline readings were taken, the products were applied and measurements taken again after 2, 4, and 6 hours. Vehicle C (Table 8) is a proprietary, highly-moisturizing oil-in-water emulsion.

### Example 7

### Long Term Benefits of KMI

The long term benefits of KMI were determined in a vehicle-controlled, double-blind, 8-week clinical study. Fifteen panelists applied Vehicle C to their faces in the mornings and evenings during the study duration, and 15 different panelists applied 1× KMI in Vehicle C. Product applications occurred after they had cleansed with their usual cleanser. The products were not applied on the morning of the days for which measurements were taken. Skin condition was measured or evaluated by expert graders at the beginning of the study (i.e. baseline), and after 4 and 8 weeks product use. Self-assessment questionnaires as to their skin condition were completed by the panelists after 2, 4, and 8 weeks of product use.

Cheek and neck moisture was evaluated using impedance measurements with the Nova Dermal Phase Meter. Firmness was evaluated using a Hargens ballistometer, a device that evaluates the elasticity and firmness of the skin by dropping a small body onto the skin and recording its first two rebound peaks. As firmness and elasticity increase, the ratio of the magnitude of the second peak to the first will increase. Clarity was evaluated using a Minolta Chromameter, which measures the total light reflected from the skin compared to the amount of red and brown/yellow light. These measurements were mathematically analyzed to determine the clarity of the skin, as Clarity=L*/(a*²+b*²)^{1/2}. Dryness was determined by an expert grader using a calibrated visual analog scale from 1 to 10. Surface fine lines were counted by expert graders, and the severity of the lines scored according to a modification of the Packman-Gans method (Packman and Gans, 1978) Canthus wrinkles were quantified by computer-assisted image analysis of negative Silflo replicas, and skin softness/suppleness was evaluated using the Gas Bearing Electrodynometer, an instrument that measures the stress/strain properties of the skin.

The instrument and expert grading results are presented in Table 13, and are recorded as % increases vs. baseline values. For all skin conditions, there was a steady increase after 4 and 8 weeks in the vehicle-treated group, but there were even greater increases for most of the measurements in the group that used the same vehicle containing 1× KMI. These differences between the two treatment groups were statistically significant. The exceptions were for skin dryness and skin clarity, for which benefits were maximal for vehicle and vehicle containing KMI. This study demonstrates that chronic anti-aging benefits can result from a daily use of a good acute moisturizer, but that KMI surprisingly and unexpectedly adds considerably to those benefits.

TABLE 13
Effects of KMI on Skin Condition During an 8-Week Treatment
Period, as Determined Using Instruments or Expert Graders

| | % Increase vs. Baseline | | | |
|---|---|---|---|---|
| | Vehicle C | | 1X KMI in Vehicle C | |
| Skin Condition | 4 Weeks | 8 Weeks | 4 Weeks | 8 Weeks |
| Cheek Moisture | 20.6 | 33.5 | 35.5* | 52.1* |
| Neck Moisture | 27.9 | 36.5 | 38.2* | 55.4* |
| Firmness | 12.1 | 24.4 | 20.5* | 30.2* |
| Softness/Suppleness | 22.2 | 32.2 | 30.1 | 45.3* |
| Canthus Wrinkles | 17.2 | 28.4 | 30.3* | 51.2* |
| Clarity | 4.8 | 8.5 | 6.3 | 12.1 |
| Surface Fine Lines | 18.1 | 29.2 | 30.3* | 45.0* |
| Dryness | 32.7 | 51.0 | 38.5 | 59.4 |

*Values with asterisks are statistically different from Vehicle C at p ≦ 0.01.

The results of the panelist self assessment of their skin condition are presented in Table 14. For both treatments and for all conditions, improvements were seen by the panelists as early as 2 weeks, and there was a steady increase during the 8-week study in the percentage of the panelists who assessed their skin condition to be “most improved” (i.e. top box on a 5 point scale). All improvements were evident for the highly moisturizing Vehicle C, but in all cases, the improvements were even greater for Vehicle C that contained 1× KMI. Thus, the panelists, expert graders, and instruments all indicated that KMI is highly effective as a chronic anti-aging blend.

TABLE 14
Effects of KMI on Panelist Self Assessment of Their Skin
Condition During an 8-Week Treatment Period

| | % of Panelists Perceiving | | | | | |
|---|---|---|---|---|---|---|
| | Increased Improved Skin Condition* | | | | | |
| | Vehicle C | | | 1X KMI in Vehicle C | | |
| Skin | 2 | 4 | 8 | 2 | 4 | |
| Condition | Weeks | Weeks | Weeks | Weeks | Weeks | 8 Weeks |
| Dryness^{A} | 53.3 | 66.7 | 86.7 | 60.0 | 80.0 | 100.0 |
| Smoothness^{B} | 46.7 | 60.0 | 80.0 | 60.0 | 73.3 | 100.0 |
| Lines and | 6.7 | 26.7 | 60.0 | 13.3 | 53.3 | 73.3 |
| Wrinkles | | | | | | |
| Firmness^{C} | 6.7 | 46.7 | 66.7 | 26.7 | 66.7 | 80.0 |
| Softness^{D} | 33.3 | 46.7 | 73.3 | 53.3 | 66.7 | 86.7 |
| Healthy | 13.3 | 26.7 | 46.7 | 20.3 | 33.3 | 66.7 |
| Glow^{E} | | | | | | |
| Elasticity^{F} | 26.7 | 53.3 | 66.7 | 26.7 | 73.3 | 86.7 |
| Looks | 13.3 | 46.7 | 73.3 | 20.0 | 66.7 | 86.7 |
| Younger^{G} | | | | | | |
| Looks | 80.0 | 100.0 | 100.0 | 86.7 | 100.0 | 100.0 |
| Healthier^{H} | | | | | | |
| *Fifteen panelists in each of the treatment cells participated in the study. After 2, 4, and 8 weeks of product use, the panelists rated their skin condition | | | | | | |
| # on a 5-point scale which compared the condition at the start of the study. The scale ranged from the assessed parameter being much less improved, somewhat less | | | | | | |
| # improved, no change, somewhat greater improved, and much greater improved. The values represent the percent of panelists who perceived much greater improvement | | | | | | |
| # at the given point in time. | | | | | | |

All of the compositions and/or methods and/or apparatus disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and/or apparatus and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,720,963
U.S. Pat. No. 6,495,126
E. Packman and E. Gans, J. Soc. Cosmetic Chem., 29:70, 1978.
Schiltz et al., J. Investig. Dermatology, 87:663-667, 1986.

1. A composition comprising:
(a) at least one regulator of lipid metabolism;
(b) at least one regulator of polysaccharide metabolism;
(c) at least one regulator of cellular protein metabolism; and
(d) at least one regulator of nucleic acid metabolism,
wherein the composition is formulated as a cosmetic blend.

2. The composition of claim 1, wherein the composition is comprised in a cosmetic vehicle.

3. The composition of claim 2, wherein the cosmetic vehicle comprises an emulsion, a cream, a lotion, a solution, an anhydrous base, a gel, or an ointment.

4. The composition of claim 3, wherein the emulsion is an oil in water emulsion or a water in oil emulsion.

5. The composition of claim 3, wherein the solution is an aqueous solution or hydro-alcoholic solution.

6. The composition of claim 3, wherein the anhydrous base is a lipstick or a powder.

7. The composition of claim 1, wherein the composition is comprised in an anti-aging product or a moisturizing product.

8. The composition of claim 1, wherein the composition is adapted for application at least once a day during use.

9. The composition of claim 1, wherein the composition is adapted for application at least twice a day during use.

10. The composition of claim 1, wherein the at least one regulator of lipid metabolism is selected from the group consisting of sodium citrate, linoleic acid, linolenic acid, biotin, glucose, sodium acetate, mevalonic acid, and serine, or derivatives thereof.

11. The composition of claim 1, wherein the at least one regulator of polysaccharide metabolism is selected from the group consisting of galactosamine, glucosamine, xylose, and magnesium chloride, or derivatives thereof.

12. The composition of claim 1, wherein the at least one regulator of cellular protein metabolism is an amino acid, or derivatives thereof.

13. The composition of claim 12, wherein the amino acid is a non-essential amino acid, or derivatives thereof.

14. The composition of claim 13, wherein the non-essential amino acid is selected from the group consisting of arginine, histidine, isoleucine, leucine, lysine, methionine, phenylalanine, threonine, tryptophan, and valine, or derivatives thereof.

15. The composition of claim 12, wherein the amino acid is selected from the group consisting of serine, aspartic acid, glutamic acid, asparagine, glutamine, alanine, tyrosine, cysteine, glycine, and proline, or derivatives thereof.

16. The composition of claim 1, wherein the at least one regulator of nucleic acid metabolism is selected from the group consisting of sodium bicarbonate, aspartic acid, sodium phosphate, niacin, glutamine, and glucose, or derivatives thereof.

17. The composition of claim 1, wherein the composition comprises from about 0.001% to about 5.0% of at least one regulator of lipid metabolism.

18. The composition of claim 1, wherein the composition comprises from about 0.001% to about 5.0% of at least one regulator of polysaccharide metabolism.

19. The composition of claim 1, wherein the composition comprises from about 0.001% to about 5.0% of at least one regulator of cellular protein metabolism.

20. The composition of claim 1, wherein the composition comprises from about 0.001% to about 5.0% of at least one regulator of cellular protein metabolism.

21. A method of treating or preventing aged or damaged skin comprising topical application of a composition comprising:
(a) at least one regulator of lipid metabolism;
(b) at least one regulator of polysaccharide metabolism;
(c) at least one regulator of cellular protein metabolism; and
(d) at least one regulator of nucleic acid metabolism,
wherein the application of the composition treats or prevents aged or damaged skin.

22. The method of claim 21, wherein the composition is chemically compatible.

23. The method of claim 21, wherein the damaged skin comprises nutritionally compromised skin or environmentally damaged skin.

24. The method of claim 23, wherein the environmentally damaged skin comprises skin damaged by u.v. light, chronic sun exposure, environmental pollutants, chemicals, disease pathologies, or smoking.

25. The method of claim 21, wherein the composition is further defined as a cosmetic composition.

26. The method of claim 21, wherein the composition is comprised in a cosmetic vehicle.

27. The method of claim 26, wherein the cosmetic vehicle comprises an emulsion, a cream, a lotion, a solution, an anhydrous base, a gel, or an ointment.

28. The method of claim 27, wherein the emulsion is an oil in water emulsion or a water in oil emulsion.

29. The method of claim 27, wherein the solution is an aqueous solution or hydro-alcoholic solution.

30. The method of claim 27, wherein the anhydrous base is a lipstick or a powder.

31. The method of claim 21, wherein the composition is comprised in an anti-aging product or a moisturizing product.

32. The method of claim 21, wherein the composition is applied at least once a day.

33. The method of claim 32, wherein the composition is applied at least twice a day.

34. The method of claim 21, wherein at least one regulator of lipid metabolism is selected from the group consisting of sodium citrate, linoleic acid, linolenic acid, biotin, glucose, sodium acetate, mevalonic acid, and serine, or derivatives thereof.

35. The method of claim 21, wherein at least one regulator of polysaccharide metabolism is selected from the group consisting of galactosamine, glucosamine, xylose, and magnesium chloride, or derivatives thereof.

36. The method of claim 21, wherein at least one regulator of cellular protein metabolism is an amino acid, or derivatives thereof.

37. The method of claim 36, wherein the amino acid is a non-essential amino acid, or derivatives thereof.

38. The method of claim 37, wherein the non-essential amino acid is selected from the group consisting of arginine, histidine, isoleucine, leucine, lysine, methionine, phenylalanine, threonine, tryptophan, and valine, or derivatives thereof.

39. The method of claim 36, wherein the amino acid is selected from the group consisting of serine, aspartic acid, glutamic acid, asparagine, glutamine, alanine, tyrosine, cysteine, glycine, and proline, or derivatives thereof.

40. The method of claim 21, wherein at least one regulator of nucleic acid metabolism is selected from the group consisting of sodium bicarbonate, aspartic acid, sodium phosphate, niacin, glutamine, and glucose, or derivatives thereof.

41. The method of claim 21, wherein the composition comprises from about 0.001% to about 5.0% of at least one regulator of lipid metabolism.

42. The method of claim 21, wherein the composition comprises from about 0.001% to about 5.0% of at least one regulator of polysaccharide metabolism.

43. The method of claim 21, wherein the composition comprises from about 0.001% to about 5.0% of at least one regulator of cellular protein metabolism.

44. The method of claim 21, wherein the composition comprises from about 0.001% to about 5.0% of at least one regulator of cellular protein metabolism.
